(19) United States
(12) Patent Application Publication
(10) Pub. No.: US 2025/0176944 A1
(43) Pub. Date: Jun. 5, 2025

(54) A THREE DIMENSIONAL ULTRASOUND IMAGING DEVICE, RELATED SYSTEM, REFERENCE ELEMENT, AND METHOD

(71) Applicant: RIGSHOSPITALET, Copenhagen Ø (DK)

(72) Inventors: Tobias TODSEN, Copenhagen Ø (DK); Fatemeh MAKOUEI, Copenhagen Ø (DK)

(73) Assignee: RIGSHOSPITALET, Copenhagen Ø (DK)

(21) Appl. No.: 18/845,024

(22) PCT Filed: Mar. 2, 2023
(86) PCT No.: PCT/EP2023/055334

§ 371 Date: Sep. 9, 2024

(30) Foreign Application Priority Data
Mar. 9, 2022 (EP) 22161064.5

(51) Int. Cl. A61B 8/00

(52) U.S. Cl. CPC A61B 8/483

(57) ABSTRACT

A three dimensional ultrasound imaging device is provided. The three dimensional ultrasound imaging device comprises a processing unit and an interface. The processing unit is configured to obtain from an ultrasound scanning machine, via the interface, scanning data indicative of a tissue sample and a reference element. The processing unit is configured to obtain, based on the scanning data, image data, where the image data is indicative of the tissue sample and the reference element. The processing unit is configured to determine, based on the image data, a tissue sample representation.

The present disclosure pertains to the field of ultrasound imaging devices, ultrasound imaging systems, reference elements, and related methods.

### BACKGROUND

A goal in surgical oncology is to remove malignant tumors and simultaneously preserve as much healthy tissue as possible. However, it may be challenging during surgery to see a tumor margin and to increase the chance of radical operation, different perioperative techniques can be used. Currently, many hospitals use biopsies from the tumor margins which are sent to pathology department for emergent frozen sections examination. Further course of a surgery depends on the surgical pathologist's evaluations. This procedure is time-consuming, increases time in general anesthesia, and has high costs. One of the limitations of frozen sections is that only a few margins can be examined, and the afterward microscopical examination of the formalin-fixated specimen can change the surgical outcome to be non-radical.

### SUMMARY

Ultrasound is a portable and cheap imaging technique that can be used perioperatively, intraoperatively, and postoperatively to provide high resolution visualization of surgical specimens. However, it may be difficult to implement ultrasound imaging techniques and achieve a satisfying accuracy when using ultrasound imaging devices.

Accordingly, there is a need for three dimensional ultrasound imaging devices, three dimensional ultrasound imaging systems, and methods for characterizing a tissue sample and a reference element, which may mitigate, alleviate, or address the shortcomings existing and may provide improved ultrasound imaging with improved visualization and improved accuracy.

A three dimensional ultrasound imaging device is provided. The three dimensional ultrasound imaging device comprises a processing unit and an interface. The processing unit is configured to obtain, from an ultrasound scanning machine, via the interface, scanning data indicative of a tissue sample and a reference element. The processing unit is configured to obtain, based on the scanning data, image data, where the image data is indicative of the tissue sample and the reference element. The processing unit is configured to determine, based on the image data, a tissue sample representation.

Further, a three dimensional ultrasound imaging system is disclosed. The three dimensional ultrasound imaging system comprises an ultrasound scanning machine comprising an ultrasound scanning probe. The three dimensional ultrasound imaging system comprises a three dimensional ultrasound imaging device comprising a processing unit, and an interface. The three dimensional ultrasound imaging system comprises a reference element. The processing unit is configured to obtain, from the ultrasound scanning machine, via the interface, scanning data indicative of a tissue sample and the reference element. The processing unit is configured to obtain, based on the scanning data, image data, where the image data is indicative of the tissue sample and the reference element. The processing unit is configured to determine a tissue sample representation based on the image data.

Further, a method, performed by a three dimensional ultrasound imaging device, for characterizing a tissue sample and a reference element is disclosed. The method comprises obtaining, based on the image data, positioning data of the reference element. The method comprises obtaining, based on the scanning data, image data, where the image data is indicative of the tissue sample and the reference element. The method comprises determining a tissue sample representation based on the image data.

The disclosed imaging device, reference element, related method, and system may provide improved ultrasound imaging, such as improved three dimensional ultrasound imaging, with improved visualization and improved accuracy. The present disclosure may provide an improved tissue sample representation with improved accuracy and in turn provide improved accuracy of assessment of resection margins of a tissue sample. For example, by providing the tissue sample representation, such as three dimensional tissue sample representation, the present disclosure may improve the visualization of an ex-vivo tissue sample, such as three dimensional visualization. The tissue sample representation may therefore provide information about e.g., resection margins of the tissue sample to a user (such as a surgeon) during surgery. In turn, the present disclosure may provide a faster feedback to a surgeon during surgery e.g., when removing tissue sample comprising a malignant tumor, and avoid the waiting time for analyzing the tissue sample by the pathology department before having information on the removed tissue sample.

For example, the present disclosure may provide precise information of a tissues sample (such as a surgical specimen) after formalin fixation. In other words, in a second stage, after the formalin fixation of the tissue sample, volume rendering of the tissue sample and/or calculating a tumor dimensions and/or margins by using scanning data may provide the pathologist with a quick and functional priori knowledge before a slicing procedure of the tissue sample. Further, the present disclosure may eliminate redundant cuts, e.g., when pathologists are to slice a tissue sample to analyze the cells of each slice. The pathologists may be provided with knowledge about the structure of the tissue sample prior to slicing, which may reduce the number of cuts, e.g., in healthy tissue.

It may be appreciated that the present disclosure provides a point-of-care imaging that may be used in an operating room and/or in a pathology laboratory. The present disclosure therefore provides a less cumbersome, faster, and simpler tissue sample representation. This may for example reduce operation time and improve the accuracy when assessing resection margins and removing tumors. Further, the present disclosure may improve the assessment of a direction of slicing of a tissue sample and a position of slicing of a tissue sample. It may be possible from the tissue sample representation to determine a direction slicing of a tissue sample and a position of slicing of a tissue sample.

Further, an advantage of the present disclosure is that the imaging device is more versatile and may be used with any ultrasound scanning machines available, such as an ultrasound scanning machine available in an operating room. For example, no add-ons may be needed to the ultrasound scanning machines and no changes in the setup of the ultrasound scanning machines.

Further, a reference element for a three dimensional ultrasound scanning system is disclosed. In other words, the reference element is for a three dimensional ultrasound scanning system as disclosed herein. The reference element comprises a three-dimensional geometrical structure. Optionally, the reference element comprises a three-dimensional grid structure. The grid structure is configured to be used as a reference when performing three-dimensional ultrasound imaging.

An advantage of the present reference element is that it allows to improve the accuracy of determination of a tissue sample representation when performing three dimensional ultrasound imaging.

### BRIEF DESCRIPTION OF THE DRAWINGS

The above and other features and advantages of the present disclosure will become readily apparent to those skilled in the art by the following detailed description of examples thereof with reference to the attached drawings, in which:

FIG. 1 schematically illustrates an exemplary three dimensional imaging system according to the present disclosure, comprising a three dimensional imaging device, an ultrasound scanning machine, and a reference element according to the present disclosure,

FIGS. 2A-B are flow diagrams of an exemplary method according to the present disclosure,

FIG. 3 shows an example three dimensional ultrasound imaging scenario applying the technique disclosed herein,

FIG. 4 shows an example three dimensional ultrasound imaging scenario applying the technique disclosed herein,

FIG. 5 shows an example reference element according to the present disclosure,

FIG. 6 shows a photography of an example tissue sample,

FIG. 7 schematically illustrates an example three dimensional imaging system according to the present disclosure seen from above, and

FIG. 8 schematically illustrates a perspective view of an example three-dimensional imaging system according to the present disclosure.

### DETAILED DESCRIPTION

Various examples and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the examples. They are not intended as an exhaustive description of the disclosure or as a limitation on the scope of the disclosure. In addition, an illustrated example needs not have all the aspects or advantages shown. An aspect or an advantage described in conjunction with a particular example is not necessarily limited to that example and can be practiced in any other examples even if not so illustrated, or if not so explicitly described.

The figures are schematic and simplified for clarity, and they merely show details which aid understanding the disclosure, while other details have been left out. Throughout, the same reference numerals are used for identical or corresponding parts.

A three dimensional (3D) ultrasound imaging device is disclosed. The three dimensional ultrasound imaging device may be seen as a device configured to provide a three dimensional (3D) ultrasound image, such as a three dimensional representation, of a sample, such as a tissue sample and/or a reference element. The three dimensional ultrasound imaging device may be seen as an electronic device, such as a computer device and/or a server device. In other words, the three dimensional ultrasound imaging device may be seen as an electronic device for three dimensional ultrasound imaging.

The three dimensional ultrasound imaging device comprises a processing unit and an interface.

The processing unit is configured to obtain, from an ultrasound scanning machine, via the interface, scanning data indicative of, such as representing, a tissue sample and a reference element. To obtain scanning data may comprise to retrieve and/or receive the scanning data from the ultrasound scanning machine. In other words, the processing unit is configured to obtain from an ultrasound scanning machine, via the interface, scanning data of a tissue sample and reference element scanned by the ultrasound scanning machine. The scanning data may be indicative of part of the tissue sample and/or part of the reference element. The three dimensional ultrasound imaging device may be connected to the ultrasound scanning machine either directly, e.g., via a cable, and/or via a network, e.g., a local network and/or a public network such as the Internet. The interface of the imaging device may comprise a wireless and/or a wired interface for connection with the ultrasound scanning machine. The ultrasound scanning machine may comprise an ultrasound scanning probe for scanning a tissue sample, such as human or animal tissue, by using ultrasound waves. The scanning data may be seen as data of an ultrasound scanning performed with the ultrasound scanning machine. The scanning data may comprise an ultrasound signal output generated by the ultrasound scanning machine. The scanning data may be seen as raw ultrasound scanning data generated by the ultrasound scanning machine. The scanning data indicative of the tissue sample and a reference element, may be seen as the scanning data comprising data representing the tissue sample and the reference element scanned by the ultrasound scanning machine.

The reference element may be seen as an element configured to provide one or more reference dimensions when performing three dimensional ultrasound imaging. In other words, the reference element may be seen as a marker.

The reference element may be scanned, using the ultrasound scanning machine, together with the tissue sample. The reference element may be positioned next to, such as proximal to, the tissue sample when performing a scanning of the tissue sample. The reference element may be positioned substantially anywhere within the field of view when performing a scanning with the ultrasound scanning machine. The reference element may comprise one or more known dimensions, such as one or more dimensions known and/or stored in the imaging device, such as stored on in a memory of the imaging device. The reference element may comprise one or more know dimensions in different planes, such as in an x-plane, a y-plane, and/or a z-plane. The reference element may also be denoted reference device. The reference element may comprise a two dimensional structure and/or a three dimensional structure.

The tissue sample may also be denoted tissue specimen, such as a surgical specimen. The tissue sample may be seen as a tissue sample from a human or an animal, such as a sample of human tissue or a sample of animal tissue. The tissue sample may for example comprise a tissue sample removed (such as resected) from a human patient by a surgeon, e.g., a surgeon performing surgical oncology. The tissue sample may be seen as an ex-vivo tissue sample from a patient. The tissue sample may be arranged in a liquid and/or a material configured to transfer ultrasound waves and/or be used with ultrasound imaging modality. For example, the tissue sample may be arranged in a buffer bath (such as water bath) when being scanned with ultrasound.

The processing unit is configured to obtain, based on the scanning data, image data, where the image data is indicative of the tissue sample and the reference element. In other words, the image data may represent at least partly the tissue sample and/or the reference element. To obtain image data may comprise to determine, to retrieve, and/or to receive the image data. For example, to obtain image data based on the scanning data, may comprise to determine image data based on the scanning data. To obtain image data may comprise to convert the scanning data into image data. To obtain image data may comprise to record video data (such as comprising a plurality of image frames over time) based on the scanning data, such as based on an ultrasound output from the ultrasound scanning machine. The image data may comprise raw video data based on the scanning data. The image data may comprise a plurality of image frames. In other words, to obtain image data may comprise to obtain one or more image frames based on the scanning data. To obtain image data may comprise to obtain one or more image frames based on a frame rate of the image data, such as frame rate of the raw video data. In other words, the image data may be decoupled into a plurality of image frames. Each image frame of the image data may be associated with a timestamp. The image data may comprise one or more image frames of the tissue sample and/or the reference element. Each image frame may comprise a slice of the tissue sample. The processing unit may be configured to identify the images frames representing the tissue sample. The processing unit may be configured to extract part of the image data representing the tissue sample. In other words, the processing unit may be configured to extract, from the image data, a region of interest representing the tissue sample.

In one or more exemplary imaging devices, the imaging device comprises an image data acquiring device. In one or more exemplary imaging devices, the processing unit is configured to obtain the scanning data from the ultrasound scanning machine via the image acquiring device. In other words, to obtain image data may comprise to record video data, using the image acquiring device, based on the scanning data, such as based on an ultrasound output from the ultrasound scanning machine. The image acquiring device may be configured to convert the scanning data into image data. In other words, the image acquiring device may be seen as a video grabber configured to convert an ultrasound scanning signal from the ultrasound scanning machine into image data. The image acquiring device may be configured to obtain one or more image frames based on the scanning data. The image acquiring device may be configured to associate a timestamp to an image frame of the image data, such as associate a timestamp for each image frame of the image data.

In one or more exemplary imaging devices, the processing unit is configured to obtain, based on the image data, positioning data of the reference element. In other words, to obtain image data may comprise to obtain positioning data of the reference element. In other words, to obtain image data may comprise to determine positioning data of the reference element. The positioning data may comprise one or more positions of the reference element. In other words, to obtain positioning data may comprise to obtain, such as determine, one or more positions, such as position coordinates, of the reference element based on the image data. To obtain positioning data may comprise to determine one or more position coordinates of the reference element based on the one or more known dimensions of the reference element. The positioning data may be seen as a positioning reference, such as a positioning reference for the image data. In other words, the positioning data may be seen as a positioning reference for the image frames of the image data.

In one or more exemplary imaging devices, the processing unit is configured to determine, based on the positioning data, a reference coordinate system. In other words, the reference coordinate system may comprise a three dimensional coordinate system. The processing unit may be configured to determine a reference coordinate system based on the one or more position coordinates of the reference element. The reference coordinate system may be seen as a global coordinate system.

The processing unit is configured to determine, based on the image data, a tissue sample representation. In other words, the processing unit may be configured to determine, based on the image data, a three dimensional (3D) tissue sample representation. The tissue sample representation may comprise a graphical representation, such as a graphical visualization, of the tissue sample. The tissue sample representation may comprise part of or all of the scanned tissue sample. The imaging device may be configured to output, such as via a display interface of the imaging device and/or a separate display interface of a separate electronic device, a user interface comprising a plurality of user interface objects. The imaging device, such as the processing unit, may be configured to output the tissue sample representation. In other words, the imaging device, such as the processing unit, may be configured to output, such as display, a user interface comprising the tissue sample representation. The tissue sample representation may comprise a representation of the tissue sample and/or a representation of the reference element. In other words, the determination of the tissue sample representation may be based on the image data associated with (such as representative of) the tissue sample and/or the reference element. The tissue sample representation may comprise a graphical representation, such as a three dimensional graphical representation, in the reference coordinate system. The tissue sample representation may be based on the plurality of image frames. In other words, to determine the tissue sample representation may be based on one or more image frames of the plurality of image frames. The tissue sample representation may comprise one or more dimensions of the tissue sample. In other words, the processing unit may be configured to determine one or more dimensions of the tissue sample. For example, the processing unit may be configured to output, such as display, via the interface, the one or more dimensions to a user of the imaging device. The one or more dimensions may be indicative of a size of a first part (such as a size of a tumor), a size of the second part, a size of a resection margin, and/or a size of the reference element.

The processing unit may be configured to extract part of the image data representing the tissue sample and determine the tissue sample representation based on the extracted part of the image data. In other words, the processing unit may be configured to determine the tissue sample representation, from the image data, a region of interest representing the tissue sample.

The processing unit may be configured to determine the tissue sample representation based on an image frame, such as a voxel, e.g., a voxel frame. To determine the tissue sample representation may comprise to determine one or more dimensions of the tissue sample, e.g., based on the image data, such as based on the position of the reference element and/or the known dimensions of the reference element.

In one or more exemplary imaging devices, the processing unit is configured to determine, for each image frame of the plurality of image frames, based on the reference coordinate system (such as based on the positioning data), a position associated with the image frame for provision of a set of positions for the plurality of image frames. In other words, the processing unit may be configured to determine one or more position coordinates associated with the image frame. In one or more exemplary imaging devices, the processing unit is configured to determine, for one or more image frame of the plurality of image frames, based on the reference coordinate system, a position associated with each of the one or more image frames for provision of a set of positions for the one or more image frames. In one or more exemplary imaging devices, the processing unit is configured to determine, for each image frame of the image frames representing the tissue sample, based on the reference coordinate system, a position associated with the image frame for provision of a set of positions for the image frames representing the tissue sample. In other words, the processing unit may be configured to position each image frame of the plurality of image frames such that the position of each image frame corresponds to the true position of the image frame. For example, the processing unit may be configured to determine, for each voxel (such as voxel frame) of a plurality of voxels, based on the reference coordinate system, a position associated with the voxel for provision of a set of positions for the plurality of voxels. In one or more exemplary imaging devices, the processing unit is configured to determine, based on the positioning data and/or the reference coordinate system, a distance between the images frames, such as a distance between two image frames.

The processing unit may be configured to determine a first position (such as a first position coordinate) for a first image frame of the plurality of image frames, a second position (such as a second position coordinate) for a second image frame of the plurality of image frames, a third position (such as a third position coordinate) for a third image frame of the plurality of image frames etc.

The processing unit may be configured to calibrate the positioning of the image frames, such as calibrate the positioning of each image frame, based on the reference coordinate system. For example, the processing unit may be configured to calibrate the positioning of the image frames based on interpolation, such as spline interpolation, and based on the positioning data of the reference element and/or the reference coordinate system. For example, the processing unit may be configured to perform interpolation of the set of positions based on the positioning data of the reference element and/or the reference coordinate system.

In one or more exemplary imaging devices, the determination of the tissue sample representation is based on the set of positions. In other words, the tissue sample representation may comprise a graphical representation, such as a three dimensional graphical representation, in the reference coordinate system. To determine the tissue sample representation may comprise to determine one or more dimensions of the tissue sample, e.g., based on the set of positions. To determine the tissue sample representation may comprise to determine one or more dimensions of the tissue sample for each image frame of the plurality of image frames, e.g., based on the set of positions. For example, the determination of the tissue sample representation may comprise to perform volumetric segmentation based on the image data, such as based on each image frame of the plurality of image frames.

In one or more exemplary imaging devices, the determination of the tissue sample representation comprises to determine, based on the image data and/or based on the scanning data, a first part of the tissue sample, the first part being associated with a first type of tissue. In other words, the processing unit may be configured to determine, based on the image data, a first part of the tissue sample, the first part being associated with a first type of tissue. For example, the processing unit may be configured to determine, based on the image data, a first type of tissue of the first part. In other words, the processing unit may be configured to determine that a first part of the tissue sample has a first property. For example, the processing unit may be configured to determine that a first part of the tissue sample has a first volumetric weight, e.g., based on the scanning data, such as based on the ultrasound signal. For example, different tissue types may have different acoustic impedance. Therefore, ultrasound waves hitting different tissue types and returning echoes would have different characteristics. For instance, a first part of a first tissue type (such as tumorous tissue) and a second part of a second tissue type (such as healthy tissue) may have different intensities and different texture in a tissue sample representation. It may therefore be possible to delineate which part of the tissue sample is a first part of a first type of tissue (such as tumorous tissue) and which part is a second part of a second type of tissue (such as healthy tissue).

For example, the determination of the tissue sample representation may comprise to delineate the first part and/or the second part. For example, the determination of the tissue sample representation volume may comprise to label the parts associated with the first part with a first label and/or the parts associated with the second part with a second label. The determination of the tissue sample representation may comprise to provide a three dimensional matrix associated with the first part (such as tumor tissue) and/or the second part (such as healthy tissue).

For example, the processing unit may be configured to determine that a first part of the tissue sample has a different volumetric weight from the volumetric weight of the remaining part of the tissue sample, such as a second part. The first type of tissue of the first part may for example comprise tissue type being a tumor, such as tumor type. In other words, the first part may be a tumorous part of the tissue sample. The first type of tissue of the first part may for example comprise tissue type being malignant tissue, such as a malignant type, e.g., a malignant tumor. In other words, the first part may be a malignant tissue part of the tissue sample. The first type of tissue of the first part may for example comprise tissue type being benign tissue, such as a benign type, e.g., a benign tumor. In other words, the first part may be a benign tissue part of the tissue sample.

In one or more exemplary imaging devices, the determination of the tissue sample representation comprises to determine, based on the image data, a second part of the tissue sample, the second part being associated with a second type of tissue. In other words, the processing unit may be configured to determine, based on the image data, a second part of the tissue sample, the second part being associated with a second type of tissue.

In other words, the processing unit may be configured to determine, based on the image data, a second part of the tissue sample, the second part being associated with a second type of tissue. For example, the processing unit may be configured to determine, based on the image data, a second type of tissue of the second part. In other words, the processing unit may be configured to determine that a second part of the tissue sample has a second property. For example, the processing unit may be configured to determine that a second part of the tissue sample has a second volumetric weight, e.g., based on the scanning data, such as based on the ultrasound signal. For example, the processing unit may be configured to determine that a second part of the tissue sample has a different volumetric weight from the volumetric weight of the remaining part of the tissue sample, such as the first part. The second type of tissue of the second part may for example comprise tissue type being healthy, such as healthy type. In other words, the second part may be a healthy part of the tissue sample, such as a healthy tissue part.

In one or more exemplary imaging devices, the determination of the tissue sample representation is based on the first part of the tissue sample and/or the second part of the tissue sample. In other words, the tissue sample representation comprises a representation of the first part and/or a representation of the second part.

In one or more exemplary imaging devices, the processing unit is configured to determine, based on the set of positions, a volume of the tissue sample.

In one or more exemplary imaging devices, the processing unit is configured to determine, based on the set of positions, a first volume of the first part and/or a second volume of the second part. To determine a first volume of the first part may comprise to determine one or more first dimensions of the first part. To determine a second volume of the second part may comprise to determine one or more second dimensions of the second part. In one or more exemplary imaging devices, the processing unit is configured to determine the first volume and/or the second volume based on the volumetric weight of the first part and/or the volumetric weight of the second part, such as the volumetric weight of the first type of tissue and/or the volumetric weight of the second type of tissue. For example, the processing unit may be configured to determine a first volume of the first part being a tumor. The first volume and/or the second volume may be expressed in liters (L). The first volume may be seen as a first size and the second volume may be seen as a second size.

The determination of the first volume and/or the second volume may comprise to determine, for each image frame of the plurality of image frames, an area of the tissue sample. The determination of the first volume and/or the second volume may comprise to determine, for each image frame of the plurality of image frames, an area of the first part and/or an area of the second part. The determination of the first volume and/or the second volume may comprise to sum the areas of the plurality of frames over an extend of the tissue sample. In other words, the determination of the first volume and/or the second volume may comprise to sum the areas representing the tissue sample for all the image frames (such as slices) representing the tissue sample. For example, the determination of the first volume and/or the second volume may comprise to sum the areas representing the tissue sample from the first image frame representing the tissue sample to the last image frame representing the tissue sample. For example, the determination of the first volume and/or the second volume may comprise to delineate the first part and/or the second part. For example, the determination of the first volume and/or the second volume may comprise to label the parts associated with the first part with a first label and/or the parts associated with the second part with a second label. The determination of the first volume and/or the second volume may comprise to provide a three dimensional matrix associated with the first part (such as tumor tissue) and/or the second part (such as healthy tissue). For example, the determination of the first volume and/or the second volume may comprise to perform three dimensional integration based on the three dimensional matrix. Alternatively and/or additionally, the determination of the first volume and/or the second volume may comprise to determine an area on a sample of image frames and then to estimate the whole volume based on the sample of image frames. This may provide a faster determination of the first volume and/or the second volume.

In one or more exemplary imaging devices, the determination of the tissue sample representation is based on the first volume of the first part and/or the second volume of the second part.

In one or more exemplary imaging devices, the processing unit is configured to determine, based on the tissue sample representation, a resection margin of the tissue sample. The processor circuitry may be configured to determine a resection margin of the tissue sample based on the first volume and/or the second volume. For example, a resection margin may be determined by subtracting the first volume from the second volume. A resection margin may be a resection margin of the first part and/or the second part. The resection margin itself may be formed by the second part. The determination of the resection margin may comprise to determine whether a resection margin is present or not, such as whether a resection margin exists or not. The processor circuitry may be configured to determine a distance from an outer surface of a second part of the tissue sample to an outer surface of a first part of the tissue sample. In other words, the processing unit may be configured to determine whether a resection margin of the tissue sample is present or not. The processing unit may be configured to determine whether the second part (such as second part of second type of tissue) encapsulates (such as encircles and/or surrounding) the first part (such as first part of first type of tissue) or not. When it is determined that the second part encapsulates (such as encircles) the first part, it is determined that a resection margin is present. When it is determined that the second part does not encapsulate (such as encircle) the first part, it is determined that no resection margin is present or at least not encapsulating the whole first part.

A resection margin of a tissue sample may be seen as a margin in the tissue sample of a second type of tissue around a first type of tissue. In other words, a resection margin may be seen as a second layer of the second type of tissue, such as a layer of healthy tissue, surrounding a first layer and/or a core of the first type of tissue, such as a layer and/or core of tumorous tissue.

A resection margin of a tissue sample may be different depending on the position on the tissue sample where the resection margin is determined. When performing resections of tissue samples from a subject, it may be desirable to have a margin (also called resection) of healthy tissue all the way around a tumor, such as encapsulating the tumor. This is to make sure that the whole tumor was removed and that the tumor has not spread into more tissue. In other words, it may be desirable to have the second part (resection margin) of the second type of tissue (healthy tissue) all the way around the first part of the first type of tissue (tumor). The determination of the resection margin may comprise to determine a type of resection margin, such as a safe resection margin (e.g., larger than 1 cm), a close margin (e.g., in the range 1 cm to 0.5 cm), and/or a positive margin (e.g., smaller than 0.5 cm).

In one or more exemplary imaging devices, the reference element comprises a three-dimensional geometrical structure, such as a three-dimensional grid structure. The reference element may comprise three-dimensional geometrical structures with known dimensions to be used reference when scanning. The three-dimensional geometrical structures may comprise patterns of geometrical structures being easy to identify and having recognizable patterns.

In one or more exemplary imaging devices, the reference element comprises a three-dimensional grid structure. In other words, the reference element may comprise a three dimensional array structure, such as an array of squares of substantially the same size. The imaging device may have one or more dimensions of the three dimensional grid structure stored, such as one or more dimensions of the three dimensional grid structure being known by the imaging device. The three dimensional grid structure may for example comprise a total width, a side length of a square of the grid, a total length, and/or a depth and/or height. The three dimensional grid structure may be used as the reference element. By using a three dimensional grid structure as reference element, it may be easier for the processing unit to determine the positioning data. The three dimensional grid structure may provide a simpler identification of the reference element by the processing unit. By having a three dimensional grid structure it may be possible to determine and/or obtain known dimensions (such as true dimensions) of the reference element in three dimensions, such as in three directions. The reference element may be three dimensional (3D) printed reference element.

In one or more exemplary imaging devices, the tissue sample is configured to be supported on a support element. In other words, the tissue sample may be supported by the support element when performing an ultrasound scanning of the tissue sample. In one or more exemplary imaging devices, the support element comprises the reference element. In other words, the reference element may be integrated and/or embedded in the support element. The support element may be seen as support for the tissue sample, such as a support that the tissue sample is attached to or arranged on when performing an ultrasound scanning of the tissue sample. In other words, the reference element may be arranged below and/or under the tissue sample. An advantage of having the tissue sample supported on a support element where the support element comprises the reference element may be that less scanning data may have to be obtained since a smaller area has to be scanned. The processing unit may be configured to obtain, based on the scanning data, image data representing the reference element through the tissue sample arranged above the reference element.

A three dimensional ultrasound imaging system is disclosed. The three dimensional ultrasound imaging system comprises an ultrasound scanning machine comprising an ultrasound scanning probe. The three dimensional ultrasound imaging system comprises a three dimensional ultrasound imaging device comprising a processing unit, and an interface, such as a three dimensional ultrasound imaging device as disclosed herein. The three dimensional ultrasound imaging system comprises a reference element, such as a reference element as disclosed herein.

The processing unit is configured to obtain, from the ultrasound scanning machine, via the interface, scanning data indicative of a tissue sample and the reference element.

The processing unit is configured to obtain, based on the scanning data, image data, where the image data is indicative of the tissue sample and/or the reference element. The processing unit is configured to determine a tissue sample representation based on the image data.

In one or more exemplary imaging systems, the processing unit is configured to determine, based on the image data, positioning data of the reference element. In one or more exemplary imaging systems, the processing unit is configured to determine, based on the positioning data, a reference coordinate system.

In one or more exemplary imaging systems, the image data comprises a plurality of image frames, and the processing unit is configured to determine, for each image frame of the plurality of image frames, based on the reference coordinate system, a position associated with the image frame for provision of a set of positions for the plurality of image frames. In one or more exemplary systems, the determination of the tissue sample representation is based on the set of positions.

In one or more exemplary imaging systems, the determination of the tissue sample representation comprises to determine, based on the image data, a first part of the tissue sample, the first part being associated with a first type of tissue. In one or more exemplary imaging systems, the determination of the tissue sample representation comprises to determine, based on the image data, a second part of the tissue sample, the second part being associated with a second type of tissue.

In one or more exemplary imaging systems, the processing unit is configured to determine, based on the set of positions, a first volume of the first part and/or a second volume of the second part.

In one or more exemplary imaging systems, the system comprises a gripping element for attaching the ultrasound scanning probe. The gripping element may be seen as a grabber. In other words, the scanning machine may comprise a gripping element for attaching the scanning probe. In one or more exemplary imaging systems, the ultrasound scanning machine comprises a slider element for moving the ultrasound scanning probe. In other words, the scanning machine may comprise a slider element for moving the scanning probe. The ultrasound scanning probe may be mounted directly to the slider without the gripping element. In one or more exemplary imaging systems, the system comprises an arm, such as a robotic arm. The arm may comprise the gripping element and/or the slider element. The system, such as the scanning machine, may comprise a motor configured to move the slider element and thereby the scanning probe. The motor may be configured to move the slider at constant speed.

In one or more exemplary imaging systems, the processing unit is configured to control the slider element. In other words, the processing unit may be configured to control a motor of the system, such as a motor of the scanning machine. The processing unit may be configured to control the slider, such as the motor, in one or more dimensions, such as one dimension, two dimensions, and/or three dimensions.

In one or more exemplary imaging systems, the reference element comprises a three dimensional grid structure.

In one or more exemplary imaging systems, the tissue sample is configured to be supported on a support element. In one or more exemplary imaging systems, the support element comprises the reference element. In one or more exemplary imaging systems, the reference element may comprise an empty area, such as a sample area, configured to receive the tissue sample. The empty area or sample area, may be seen as an area where the reference element does not have any reference pattern, e.g., a plane surface where the tissues sample may be placed. In one or more example imaging systems, the sample area may be implemented as an opening in the reference element, e.g., where the reference element does not comprise any material, such as a hole in the reference element. The sample area may be shaped as a square. An advantage of having a reference element with an opening may be that the tissue sample may be scanned without background. The reference element may be used to gravity clamp the support, such as cork, and/or the tissue sample, e.g., to ensure that they are covered with saline solution.

A reference element for a three-dimensional ultrasound scanning system is disclosed. The reference element comprises a three dimensional grid structure. The grid structure is configured to be used as a reference when performing three dimensional ultrasound imaging. In other words, the reference element may be for a three dimensional ultrasound scanning system as disclosed herein.

A use of a reference element according to the present disclosure for a three-dimensional ultrasound scanning system is disclosed.

A method, performed by a three dimensional ultrasound imaging device, for characterizing a tissue sample and a reference element is disclosed. The method comprises obtaining, from an ultrasound scanning machine, scanning data indicative of the tissue sample and the reference element. The method comprises obtaining, based on the scanning data, image data, where the image data is indicative of the tissue sample and the reference element. The method comprises determining a tissue sample representation based on the image data.

It is to be understood that a description of a feature in relation to the imaging device(s) is also applicable to the corresponding feature in the imaging system(s), the reference element, and/or the method(s).

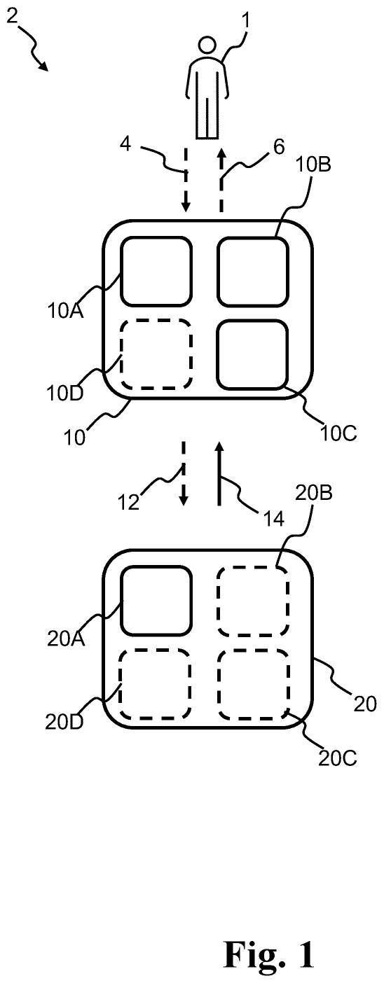

FIG. 1 schematically illustrates an exemplary system, such as a three dimensional imaging system 2, according to the present disclosure. The system 2 comprises an ultrasound scanning machine 20 comprising an ultrasound scanning probe 20A. The three dimensional ultrasound imaging system 2 comprises a three dimensional ultrasound imaging device 10 comprising a processing unit 10C, and an interface 10B (such as one or more interfaces), such as a three dimensional ultrasound imaging device according to the present disclosure. The three dimensional ultrasound imaging system 2 comprises a reference element (not shown), such as a reference element according to the present disclosure. Optionally, the imaging device 10 comprises a memory 10A. Optionally, the scanning machine 20 comprises one or more interface 20B.

The processing unit 10C is configured to obtain 14, from the ultrasound scanning machine 20, via the interface 10B, scanning data indicative of a tissue sample and a reference element.

The processing unit 10C is configured to obtain, based on the scanning data, image data, where the image data is indicative of the tissue sample and the reference element.

The processing unit 10C is configured to determine, based on the image data, a tissue sample representation.

Optionally, the imaging device 10 may be configured to output 6 (such as user output), such as via a display interface of the imaging device 10 and/or a separate display interface of a separate electronic device, a user interface comprising a plurality of user interface objects to a user 1 of the imaging device. The imaging device 10, such as the processing unit 10C, may be configured to output 6, such as via the interface 10A, the tissue sample representation to the user 1. In other words, the imaging device 10, such as the processing unit 10C, may be configured to output 6, such as display, a user interface comprising the tissue sample representation to the user 1.

Optionally, the user 1 may provide an input 4 (such as user input), such as via the interface 10A, to the imaging device 10. The determination of the tissue sample representation may be based on the input 4 from the user. The user 1 may for example select a region of interest of the tissue sample to be represented. The user 1 may for example provide an input 4 comprising one or more dimensions of the reference element.

For example, the processing unit may be configured to output, such as display, via the interface, the one or more dimensions to a user of the imaging device. The one or more dimensions may be indicative of a size of a first part (such as a size of a tumor), a size of the second part, a size of a resection margin, and/or a size of the reference element.

In one or more exemplary imaging devices, the processing unit 10C is configured to obtain, based on the image data, positioning data of the reference element. In one or more exemplary imaging systems and/or imaging devices, the processing unit 10C is configured to determine, based on the positioning data, a reference coordinate system.

In one or more exemplary imaging systems and/or imaging devices, the image data comprises a plurality of image frames, and the processing unit 10C is configured to determine, for each image frame of the plurality of image frames, based on the reference coordinate system, a position associated with the image frame for provision of a set of positions for the plurality of image frames. In one or more exemplary imaging devices, the determination of the tissue sample representation is based on the set of positions.

In one or more exemplary imaging systems and/or imaging devices, the processing unit 10C is configured to determine, based on the image data, a first part of the tissue sample, the first part being associated with a first type of tissue. In one or more exemplary imaging devices, the processing unit 10C is configured to determine, based on the image data, a second part of the tissue sample, the second part being associated with a second type of tissue.

In one or more exemplary imaging systems and/or imaging devices, the processing unit 10C is configured to determine, based on the set of positions, a first volume of the first part and/or a second volume of the second part.

In one or more exemplary imaging systems and/or imaging devices, the processing unit 10C is configured to determine, based on the tissue sample representation, a resection margin of the tissue sample.

In one or more exemplary imaging systems and/or imaging devices, the imaging device 10 comprises an image data acquiring device 10D. In one or more exemplary imaging systems and/or imaging devices, the processing unit 10C is configured to obtain 14 the scanning data from the ultrasound scanning machine 20 via the image acquiring device 10D.

In one or more exemplary imaging systems and/or imaging systems, the system 2 comprises a gripping element 20C for attaching the ultrasound scanning probe 20A. The gripping element 20C may be seen as a grabber. In other words, the scanning machine 20 may comprise a gripping element 20C for attaching the scanning probe 20A. In one or more exemplary imaging systems, the ultrasound scanning machine comprises a slider element 20D for moving the ultrasound scanning probe 20A.

In one or more exemplary imaging systems and/or imaging systems, the processing unit 10C is configured to control 12 (such as transmit one or more signals and/or instructions) the slider element 20D. In other words, the processing unit 10 may be configured to control 12 a motor of the system 2, such as a motor of the scanning machine 20.

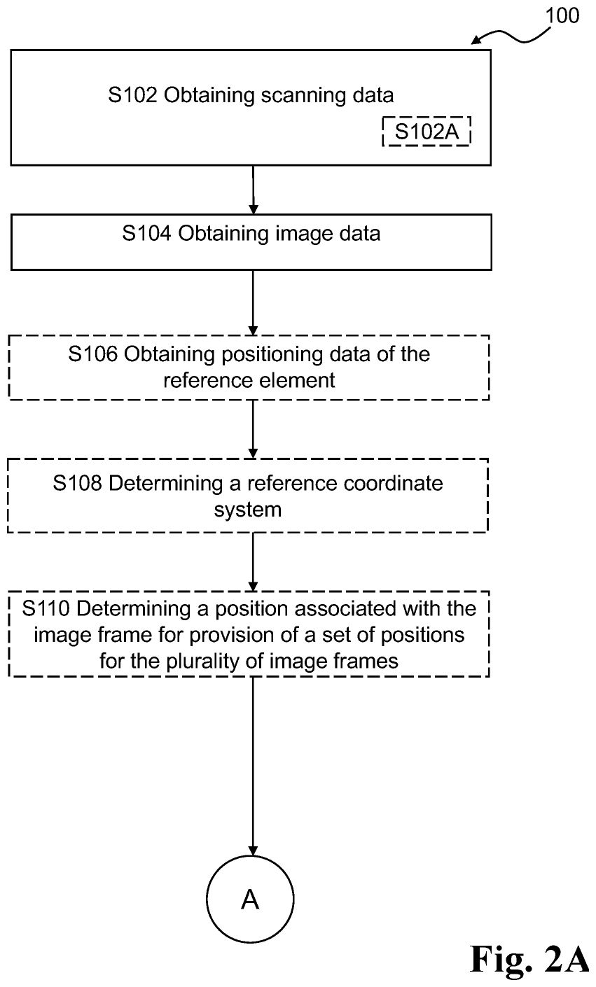

The electronic device 10 may be configured to perform any of the methods disclosed in FIGS. 2A, 2B.

The processing unit 10C is optionally configured to perform any of the operations disclosed in FIGS. 2A-2B (such as any one or more of S102A, S106, S108, S110, S112A, S112B, S114). The operations of the imaging device may be embodied in the form of executable logic routines (for example, lines of code, software programs, etc.) that are stored on a non-transitory computer readable medium (for example, memory 10A) and are executed by the processing unit 10C).

Furthermore, the operations of the imaging device 10 may be considered a method that the imaging device 10 is configured to carry out. Also, while the described functions and operations may be implemented in software, such functionality may as well be carried out via dedicated hardware or firmware, or some combination of hardware, firmware and/or software.

Memory 10A may be one or more of a buffer, a flash memory, a hard drive, a removable media, a volatile memory, a non-volatile memory, a random access memory (RAM), or other suitable device. In a typical arrangement, memory 10A may include a non-volatile memory for long term data storage and a volatile memory that functions as system memory for the processing unit 10C. The memory 10A may exchange data with the processing unit 10C over a data bus. Control lines and an address bus between the memory 10B and the processing unit 10C also may be present (not shown in FIG. 1). The memory 10B is considered a non-transitory computer readable medium.

The memory 10B may be configured to store information such as scanning data, image data, and/or tissue sample representation(s) in a part of the memory.

FIGS. 2A and 2B show a flow diagram of an exemplary method, such as a method 100. The method 100, performed by a three dimensional ultrasound imaging device, for characterizing a tissue sample and a reference element is disclosed. The method 100 comprises obtaining S102, from an ultrasound scanning machine, scanning data indicative of the tissue sample and the reference element. The method 100 comprises obtaining S104, based on the scanning data, image data, where the image data is indicative of the tissue sample and the reference element. The method 100 comprises determining S112 a tissue sample representation based on the image data.

In one or more exemplary methods, the method 100 comprises obtaining S106, based on the image data, positioning data of the reference element.

In one or more exemplary methods, the method 100 comprises determining S108, based on the positioning data, a reference coordinate system.

In one or more exemplary methods, the image data comprises a plurality of image frames, the method 100 comprises determining S110, for each image frame of the plurality of image frames, based on the reference coordinate system, a position associated with the image frame for provision of a set of positions for the plurality of image frames.

In one or more exemplary methods, the determination S112 of the tissue sample representation is based on the set of positions.

In one or more exemplary methods, the determination S112 of the tissue sample representation comprises determining S112A, based on image data, a first part of the tissue sample, the first part being associated with a first type of tissue.

In one or more exemplary methods, the method 100 comprises determining S112A, based on image data, a second part of the tissue sample, the second part being associated with a second type of tissue.

In one or more exemplary methods, the method 100 comprises determining S112B, based on the set of positions, a first volume of the first part and/or a second volume of the second part.

In one or more exemplary methods, the method 100 comprises determining S114, based on the tissue sample representation, a resection margin of the tissue sample.

In one or more exemplary methods, the method 100 comprises obtaining S102A, the scanning data from an ultrasound scanning machine via an image acquiring device.

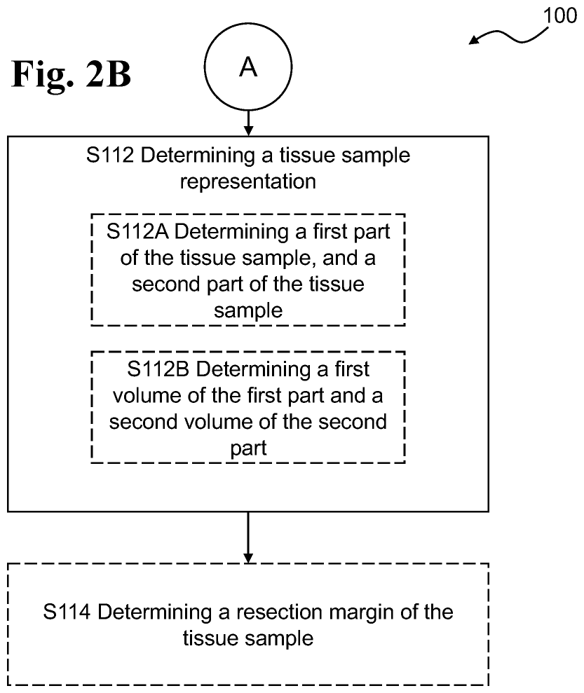

FIG. 3 shows an example three dimensional ultrasound imaging scenario applying the technique disclosed herein. FIG. 3 shows an example scenario where a three dimensional ultrasound imaging device according to the present disclosure has been used. The processing unit (such as processing unit 10C of FIG. 1) has obtained from an ultrasound scanning machine, via the interface, scanning data indicative of a tissue sample 200 and a reference element 300. The processing unit (such as processing unit 10C of FIG. 1) has obtained, based on the scanning data, image data, where the image data is indicative of the tissue sample 200 and the reference element 300. The processing unit (such as processing unit 10C of FIG. 1) has determined, based on the image data, a tissue sample representation 250. In FIG. 3, the tissue sample representation 250 is a three dimensional tissue sample representation, such as a three dimensional visualization of the tissue sample 200.

Optionally, the processing unit (such as processing unit 10C of FIG. 1) has obtained, based on the image data, positioning data of the reference element 300, and has determined, based on the positioning data, a reference coordinate system 260. The reference coordinate system 260 may be seen on FIG. 3 as a three dimensional reference coordinate system with an X-axis, a Y-axis, and a Z-axis.

Optionally, the determination of the tissue sample representation comprises to determine, based on the image data, a first part 210 of the tissue sample 200, the first part 210 being associated with a first type of tissue, and a second part 220 of the tissue sample 200, the second part 220 being associated with a second type of tissue.

Optionally, the processing unit (such as processing unit 10C of FIG. 1) is configured to determine, based on the set of positions, a first volume of the first part 210 and a second volume of the second part 220.

Optionally, the processing unit (such as processing unit 10C of FIG. 1) is configured to determine, based on the tissue sample representation 250, a resection margin 230 of the tissue sample 200.

Optionally, the reference element 300 comprises a three dimensional grid structure.

Optionally, the tissue sample 200 is configured to be supported on a support element, and wherein the support element comprises the reference element 300. In FIG. 3, the tissue sample 200 was supported on the reference element 300 when the tissue sample 200 was scanned with ultrasound.

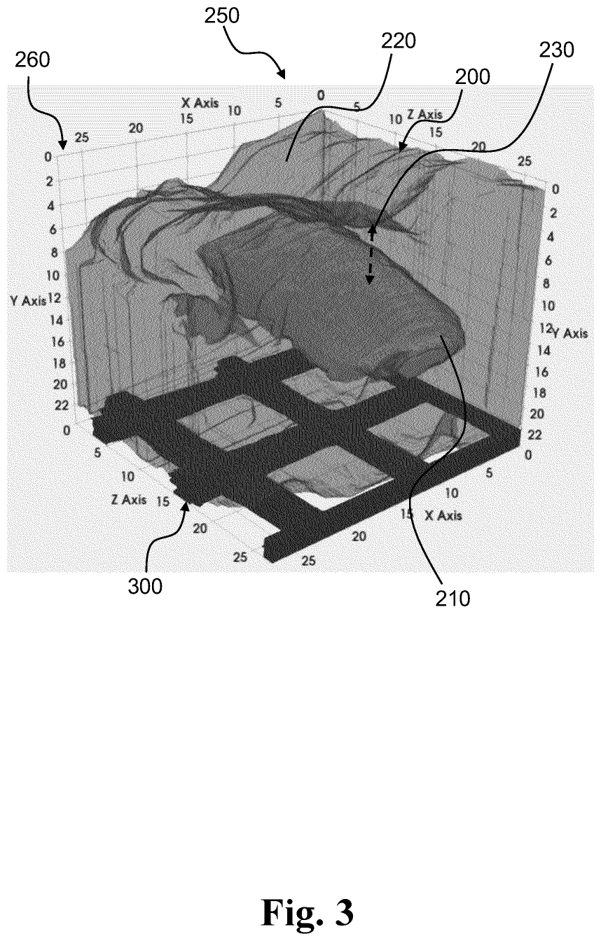

FIG. 4 shows an example three dimensional ultrasound imaging scenario applying the technique disclosed herein. FIG. 4 shows an example scenario where a three-dimensional ultrasound imaging device, such as the imaging device 10, according to the present disclosure has been used, e.g., to provide a tissue sample representation, such as the tissue sample representation of FIG. 3.

Optionally, the image data comprises a plurality of image frames. For example, the image data comprises a first image frame 240 (such as first image plane), a second image frame 242 (such as second image plane), and/or a third image frame 244 (such as third image plane). In FIG. 4, the image data comprises thirteen image frames.

The processing unit, such as processing unit 10C, may be configured to determine, for each image frame of the plurality of image frames, based on the reference coordinate system 260, a position associated with the image frame for provision of a set of positions for the plurality of image frames. Optionally, the determination of the tissue sample representation 250 is based on the set of positions.

In one or more exemplary imaging devices, the processing unit, (such as processing unit 10C of FIG. 1) is configured to determine, for one or more image frame of the plurality of image frames, based on the reference coordinate system 260, a position associated with each of the one or more image frames for provision of a set of positions for the one or more image frames. For example, the processing unit may be configured to determine a first position of the first image frame 240, a second position of the second image frame 242, and/or a third position of the third image frame 244. In one or more exemplary imaging devices, the processing unit (such as processing unit 10C of FIG. 1) is configured to determine, for each image frame of the image frames representing the tissue sample 200, based on the reference coordinate system 260, a position associated with the image frame for provision of a set of positions for the image frames representing the tissue sample 200. In other words, the processing unit may be configured to position each image frame of the plurality of image frames such that the position of each image frame corresponds to the true position of the image frame, e.g., true position when scanning the tissue sample 200. For example, the processing unit may be configured to determine, for each voxel (such as voxel frame) of a plurality of voxels, based on the reference coordinate system 260, a position associated with the voxel for provision of a set of positions for the plurality of voxels. In one or more exemplary imaging devices, the processing unit is configured to determine, based on the positioning data and/or the reference coordinate system 260, a distance between the images frames, such as a distance between two image frames, such as between the first image frame 240 and the second image frame 242.

The processing unit may be configured to determine a first position (such as a first position coordinate) for a first image frame 240 of the plurality of image frames, a second position (such as a second position coordinate) for a second image frame 242 of the plurality of image frames, a third position (such as a third position coordinate) for a third image frame 244 of the plurality of image frames etc.

The processing unit may be configured to calibrate the positioning of the image frames, such as calibrate the positioning of each image frame, based on the reference coordinate system 260. For example, the processing unit may be configured to calibrate the positioning of the image frames based on interpolation, such as spline interpolation, and based on the positioning data of the reference element 300 and/or the reference coordinate system 260. For example, the processing unit may be configured to perform interpolation of the set of positions based on the positioning data of the reference element 300 and/or the reference coordinate system 260.

To determine the tissue sample representation 250 may comprise to determine one or more dimensions of the tissue sample 200 for each image frame of the plurality of image frames, e.g., based on the set of positions. For example, the determination of the tissue sample representation 250 may comprise to perform volumetric segmentation based on the image data, such as based on each image frame of the plurality of image frames.

The determination of the first volume and/or the second volume may comprise to determine, for each image frame of the plurality of image frames, an area of the tissue sample. The determination of the first volume and/or the second volume may comprise to determine, for each image frame of the plurality of image frames, an area of the first part 210 and/or an area of the second part 220. The determination of the first volume and/or the second volume may comprise to sum the areas of the plurality of frames over an extend of the tissue sample 200. In other words, the determination of the first volume and/or the second volume may comprise to sum the areas representing the tissue sample 200 for all the image frames (such as slices) representing the tissue sample 200. For example, the determination of the first volume and/or the second volume may comprise to sum the areas representing the tissue sample 200 from the first image frame 240 representing the tissue sample to the last image frame 270 representing the tissue sample 200. For example, the determination of the first volume and/or the second volume may comprise to delineate the first part 210 and/or the second part 220. For example, the determination of the first volume and/or the second volume may comprise to label the parts associated with the first part 210 with a first label and/or the parts associated with the second part 220 with a second label. The determination of the first volume and/or the second volume may comprise to provide a three dimensional matrix associated with the first part 210 (such as tumor tissue) and/or the second part 220 (such as healthy tissue). For example, the determination of the first volume and/or the second volume may comprise to perform three dimensional integration based on the three dimensional matrix. Alternatively and/or additionally, the determination of the first volume and/or the second volume may comprise to determine an area on a sample of image frames and then to estimate the whole volume based on the sample of image frames. This may provide a faster determination of the first volume and/or the second volume.

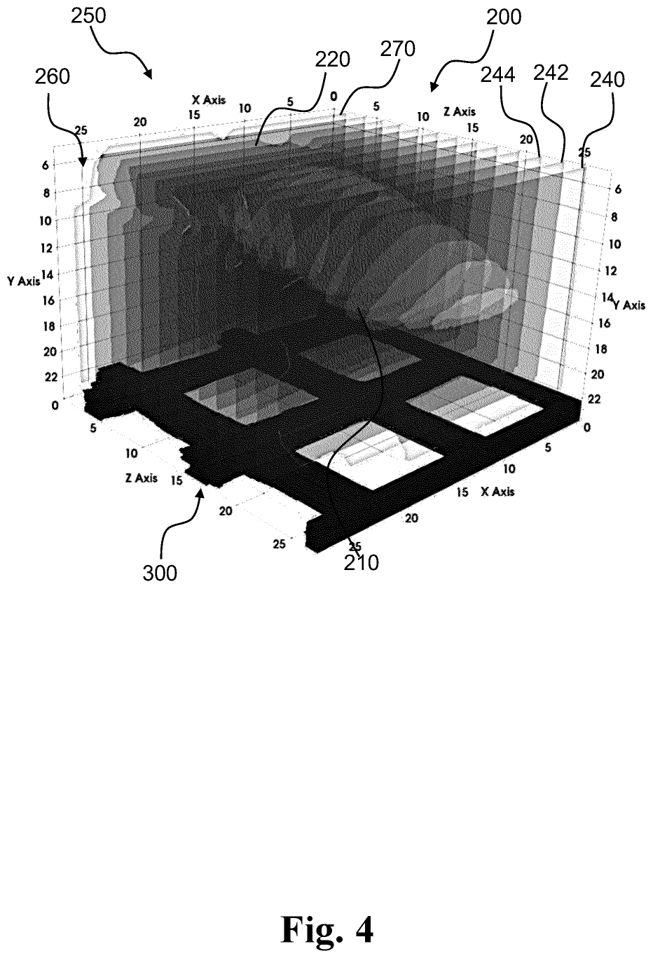

FIG. 5 shows an example reference element according to the present disclosure, such as reference element 300 of FIGS. 3-4. The reference element 300 may be seen as an element configured to provide one or more reference dimensions when performing three dimensional ultrasound imaging, such as using imaging device 10 of FIG. 1. In other words, the reference element 300 may be seen as a marker.

The reference element 300 may be scanned, using the ultrasound scanning machine, together with a tissue sample, such as tissue sample 200 of FIGS. 3-4. The reference element 300 may be positioned next to, such as proximal to, the tissue sample when performing a scanning of the tissue sample. The reference element 300 may be positioned substantially anywhere within the field of view when performing a scanning with the ultrasound scanning machine. The reference element 300 may comprise one or more known dimensions, such as one or more dimensions known and/or stored in the imaging device, such as stored on in a memory of the imaging device. The reference element 300 may comprise one or more know dimensions in different planes, such as in an x-plane, a y-plane, and/or a z-plane. The reference element 300 may also be denoted reference device. The reference element 300 may comprise a two dimensional structure and/or a three dimensional structure. In FIG. 5, the reference element 300 comprises a three dimensional grid structure, and the grid structure is configured to be used as a reference when performing three dimensional ultrasound imaging. The reference element 300 comprises a three dimensional array structure, such as an array of squares of substantially the same size. The imaging device may have one or more dimensions of the three dimensional grid structure stored, such as one or more dimensions of the three dimensional grid structure being known by the imaging device. The three dimensional grid structure may for example comprise a total width, a side length of a square of the grid, a total length, and/or a depth and/or height. The three dimensional grid structure may be used as the reference element 300. By using a three dimensional grid structure as reference element, it may be easier for the processing unit to determine the positioning data. The three dimensional grid structure may provide a simpler identification of the reference element by the processing unit. By having a three dimensional grid structure it may be possible to determine and/or obtain known dimensions (such as true dimensions) of the reference element in three dimensions, such as in three directions.

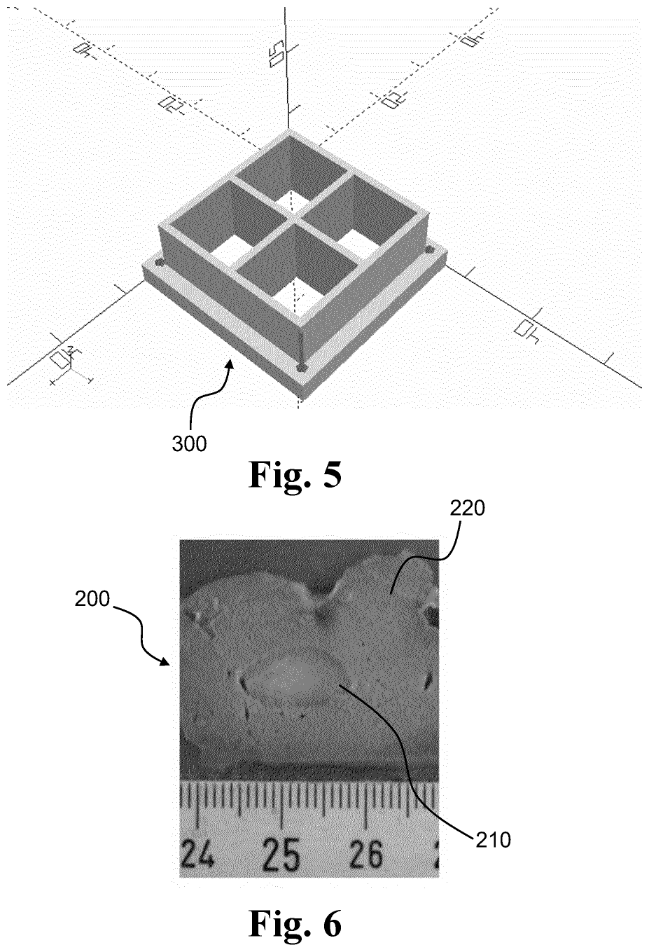

FIG. 6 shows a photography of an example tissue sample, such as the tissue sample 200 of FIGS. 4-5. The tissue sample 200 is an animal tissue sample. The tissue sample 200 has been sliced so that the photography shows a cross section of the tissue sample 200. The tissue sample 200 comprises a first part 210, the first part 210 being associated with a first type of tissue (such as tumorous tissue), and the tissue sample 200 comprises a second part 220, the second part 220 being associated with a second type of tissue. FIGS. 3-4 may comprise a tissue sample representation of the tissue sample 200 of FIG. 6.

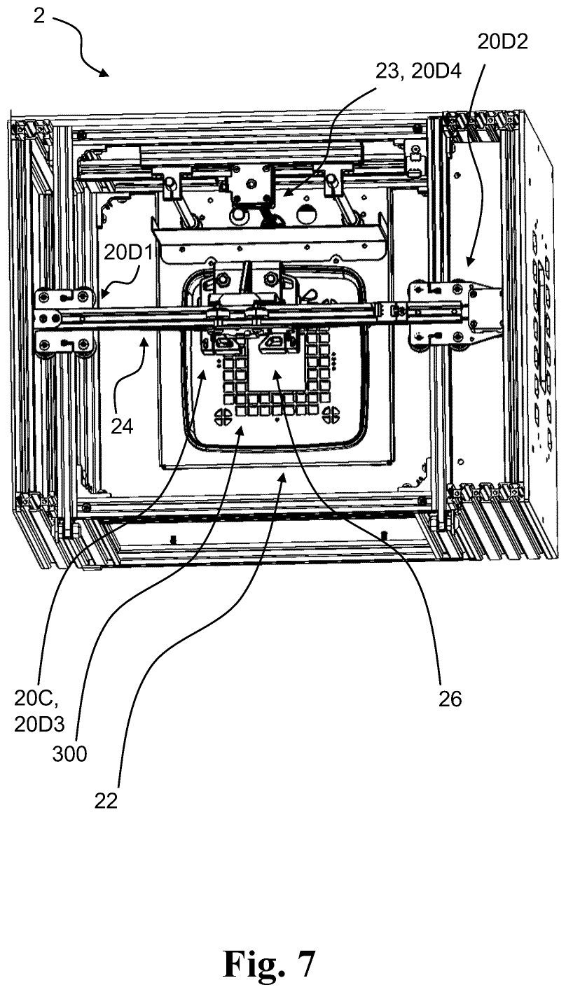

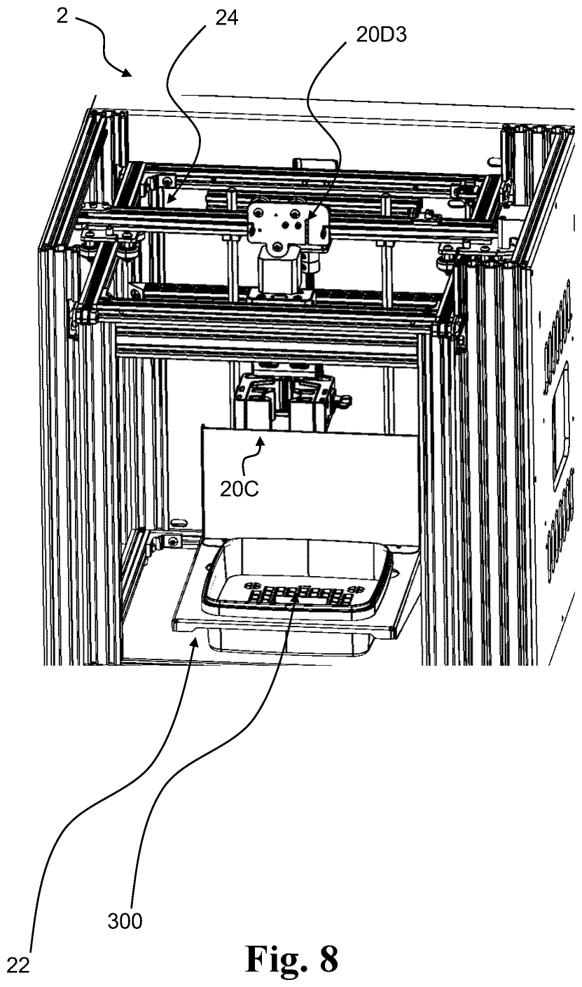

FIGS. 7-8 schematically illustrate examples of three dimensional imaging systems according to the present disclosure, such as imaging system 2. In FIG. 7 the example three dimensional imaging system is seen from above, and FIG. 8 shows a perspective view of the example three dimensional imaging system. It may be appreciated that the ultrasound scanning machine comprising the ultrasound scanning probe and the three dimensional ultrasound imaging device comprising the processing unit and interface are not shown in the figures. The imaging system shown in FIGS. 7-8 comprises a cabinet, which may be closed on all sides. For matter of illustration, the top and side cover of the cabinet are hidden. The cabinet may be configured to be connected to a ventilation system, e.g., to prevent cross contamination and/or out-gassing from the tissue sample, and to ensure that the mechanical motion system does not contaminate the air in the operating volume, such as operating theater. The cabinet may further function as operator safety, e.g., preventing pinching, clamping, burns, and access may be controlled by a monitored door, which ensures that all motion is stopped if the door is opened during scanning. The imaging system 2 may be connected to an external computer, such as a laptop, configured to act as the processing unit.

The imaging system 2 comprises a reference element 300, such as a reference element as disclosed herein. As may be seen in FIGS. 7-8, the reference element 300 is placed in a carrier element 22. It may be appreciated that the reference element 300 may have an empty area, a sample area 26, configured to receive the tissue sample. The empty area or sample area 26, may be seen as an area where the reference element does not have any reference pattern, e.g., a plane surface where the tissues sample may be placed. In one or more example imaging systems, the sample area 26 may be implemented as an opening in the reference element 300. An advantage of having a reference element with an opening may be that the tissue sample may be scanned without background. The reference element 300 may be used to gravity clamp the support, such as cork, and/or the tissue sample, e.g., to ensure that they are covered with saline solution. As may be seen on FIGS. 7-8, the sample area 26 is here squared. In one or more exemplary imaging systems, the sample area 26 may be configured to receive a support element, such as a piece of cork. In other words, the tissues sample may be pinned to the piece of cork in the sample area 26. In one or more exemplary imaging systems, the support element comprises the reference element 300 or vice versa. The carrier element 22 may comprise a container where the reference element 300 and the tissue sample are placed. The container may then be filled up with saline solution for covering the tissue sample. Preferably, the tissue sample is covered with saline solution such that there is at least 5 mm of saline solution above the highest point of the tissue sample, for example at least 10 mm above, at least 20 mm above, or at least 50 mm above. Optionally, the imaging system 2 may comprise a thermometer for measuring the temperature of the saline solution. As dimensions obtained using ultrasonic scanning are dependent on the temperature (sound propagation varies with temperature), the temperature of the saline bath where the tissue sample is placed may be measured while performing a scanning. The obtained temperature may then be logged together with the scanning data, e.g., by the processing unit. In the example of FIGS. 7-8, the reference element 300 comprises a three-dimensional grid structure. The grid structure is configured to be used as a reference when performing three dimensional ultrasound imaging, such as geometrical reference. In this example, the reference element comprises a grid structure around the sample area 26. Optionally, the reference element 300 comprises auxiliary reference structures at the corners of the reference element 300, such as at the corners of the grid structure. The auxiliary reference structures may be used for optional geometry calibration. The auxiliary reference structures may have different encapsulation than the remaining reference structures and may be identified automatically and used for global alignment.

In one or more exemplary imaging systems, the system 2 comprises a gripping element 20C for attaching the ultrasound scanning probe (not shown). The gripping element 20C may be seen as a grabber. In one or more exemplary imaging systems, the imaging system 2 comprises a slider element 20D for moving the ultrasound scanning probe. The ultrasound scanning probe may be mounted directly to the slider element 20D without the gripping element 20C. It may be appreciated that the system 2 may comprise a plurality of slider elements 20D, such as a first slider element, a second slider element, a third slider element, and/or a fourth slider element. For example, the system 2 may comprise two slider elements 20D on each side of the gripping element 20C, such as the first slider element 20D1 and the second slider element 20D2, for allowing motion/movement on an X-axis of the system, such as X-axis of the cabinet. Additionally or alternatively, the system 2 may comprise a slider element 20D, such as a third slider element 20D3, for allowing motion/movement on a Y-axis of the system, such as Y-axis of the cabinet. For example, the gripping element 20C may be mounted on the third slider element 20D3. In one or more exemplary imaging systems, the system 2 comprises a beam 24 connecting two slider elements 20D, 20D1, 20D2 on each side of the cabinet. In one or more exemplary imaging systems, the gripping element 20C may be mounted on the beam 24.

The system 2 may comprise one or more motors configured to move the slider element(s) 20D and thereby the scanning probe, e.g., in turn the gripping element 20C. The motor may be configured to move the slider at constant speed. Thereby, the system 2 is configured to move the scanning probe in the X-Y plane. For example, each slider element 20D may comprise a motor. In one or more exemplary imaging systems, the system 2 is configured to rotate the scanning probe around a Z-axis of the system, such as a Z-axis of the cabinet. In other words, the gripping element 20C and/or the third slider element 20D3 may comprise a motor, e.g., for allowing rotation around the Z-axis. In one or more exemplary imaging systems, the carrier element 22 is mounted on a slider element, such as a fourth slider element 20D4. The fourth slider element 20D4 may allow motion/movement of the carrier element 22, such as movement of the tissue sample, on a Z-axis of the system, such as Z-axis of the cabinet. It may thereby be appreciated that the system 2 may provide four degrees of freedom of movement in a Cartesian coordinate system. With these degrees of freedom, the system 2 may be capable of aligning the scanning probe relative to the tissue sample and in principle allows for scanning along any trajectory in plane, e.g., with the scanner array oriented perpendicular to the trajectory of the scanning direction. It may be appreciated that the scanning probe may comprise a scanner array internally of an array of elements, e.g., aligned with the width of the probe. When scanning, the scanning direction may have to be perpendicular to the array. In one or more exemplary imaging systems, the system 2 may be configured to scan a sample along both X and Y axis separately, e.g., for improving geometry- and feature recognition. By having a system allowing movement in 3D, the scanning probe may be positioned perpendicularly to the trajectory of the scanning direction. For example, when the tissue sample is scanned in X direction and then the Y direction, requires that the scanning probe can be rotated around the Z-axis 90 degrees.

Examples of imaging devices, systems, reference elements, and methods according to the disclosure are set out in the following items:

Item 1. A three dimensional ultrasound imaging device comprising:
a processing unit;
an interface; and
wherein the processing unit is configured to:
obtain, from an ultrasound scanning machine, via the interface, scanning data indicative of a tissue sample and a reference element;
obtain, based on the scanning data, image data, where the image data is indicative of the tissue sample and the reference element, and
determine, based on the image data, a tissue sample representation.

Item 2. The imaging device according to item 1, wherein the processing unit is configured to obtain, based on the image data, positioning data of the reference element, and to determine, based on the positioning data, a reference coordinate system.

Item 3. The imaging device according to item 2, wherein the image data comprises a plurality of image frames, and wherein the processing unit is configured to determine, for each image frame of the plurality of image frames, based on the reference coordinate system, a position associated with the image frame for provision of a set of positions for the plurality of image frames, and wherein the determination of the tissue sample representation is based on the set of positions.

Item 4. The imaging device according to any of the previous items, wherein the determination of the tissue sample representation comprises to determine, based on the image data, a first part of the tissue sample, the first part being associated with a first type of tissue, and a second part of the tissue sample, the second part being associated with a second type of tissue.

Item 5. The imaging device according to item 4, wherein the processing unit is configured to determine, based on the set of positions, a first volume of the first part and a second volume of the second part.

Item 6. The imaging device according to any of the previous items, wherein the processing unit is configured to determine, based on the tissue sample representation, a resection margin of the tissue sample.

Item 7. The imaging device according to any of the previous items, wherein the reference element comprises a three dimensional grid structure.

Item 8. The imaging device according to any of the previous items, wherein the tissue sample is configured to be supported on a support element, and wherein the support element comprises the reference element.

Item 9. The imaging device according to any of the previous items, wherein the imaging device comprises an image data acquiring device, and wherein the processing unit is configured to obtain the scanning data from the ultrasound scanning machine via the image acquiring device.

Item 10. A three dimensional ultrasound imaging system comprising:
an ultrasound scanning machine comprising an ultrasound scanning probe;
a three dimensional ultrasound imaging device comprising a processing unit and an interface;
a reference element; and
wherein the processing unit is configured to:
obtain, from the ultrasound scanning machine, via the interface, scanning data indicative of a tissue sample and the reference element;
obtain, based on the scanning data, image data, where the image data is indicative of the tissue sample and the reference element, and to determine a tissue sample representation based on the image data.

Item 11. The system according to item 10, wherein the processing unit is configured to determine, based on the image data, positioning data of the reference element, and to determine, based on the positioning data, a reference coordinate system.

Item 12. The system according to item 11, wherein the image data comprises a plurality of image frames, and wherein the processing unit is configured to, for each image frame of the plurality of image frames, determine, based on the reference coordinate system, a position associated with the image frame for provision of a set of positions for the plurality of image frames, and wherein the determination of the tissue sample representation is based on the set of positions.

Item 13. The system according to any of items 10-12, wherein the determination of the tissue sample representation comprises to determine, based on the image data, a first part of the tissue sample, the first part being associated with a first type of tissue, and a second part of the tissue sample, the second part being associated with a second type of tissue.

Item 14. The system according to item 13, wherein the processing unit is configured to determine, based on the set of positions, a first volume of the first part and a second volume of the second part.

Item 15. The system according to any of items 10-14, wherein the system comprises a gripping element for attaching the ultrasound scanning probe, and wherein the ultrasound scanning machine comprises a slider element for moving the ultrasound scanning probe.

Item 16. The system according to item 15, wherein the processing unit is configured to control the slider element.

Item 17. The system according to any of items 10-16, wherein the reference element comprises a three dimensional grid structure.

Item 18. The system according to any of items 10-17, wherein the tissue sample is configured to be supported on a support element, and wherein the support element comprises the reference element.

Item 19. A reference element for a three dimensional ultrasound scanning system, wherein the reference element comprises a three dimensional grid structure, and wherein the grid structure is configured to be used as a reference when performing three dimensional ultrasound imaging.

Item 20. A method, performed by a three dimensional ultrasound imaging device, for characterizing a tissue sample and a reference element, the method comprising:
obtaining (S102), from an ultrasound scanning machine, scanning data indicative of the tissue sample and the reference element,
obtaining (S104), based on the scanning data, image data, where the image data is indicative of the tissue sample and the reference element; and
determining (S118) a tissue sample representation based on the image data.

Item 21. The method according to item 20, wherein the method comprises:
obtaining (S106), based on the image data, positioning data of the reference element, and
determining (S108), based on the positioning data, a reference coordinate system.

Item 22. The method according to item 21, wherein the image data comprises a plurality of image frames, the method comprising:
determining (S110), for each image frame of the plurality of image frames, based on the reference coordinate system, a position associated with the image frame for provision of a set of positions for the plurality of image frames; and
where the determination (S118) of the tissue sample representation is based on the set of positions.

Item 23. The method according to any of items 20-22, wherein the method comprises:
determining (S112), based on image data, a first part of the tissue sample, the first part being associated with a first type of tissue, and a second part of the tissue sample, the second part being associated with a second type of tissue.

Item 24. The method according to item 23, wherein the method comprises:
determining (S114), based on the set of positions, a first volume of the first part and a second volume of the second part.

Item 25. The method according to any of items 20-24, wherein the method comprises:
determining (S120), based on the tissue sample representation, a resection margin of the tissue sample.

Item 26. The method according to any of items 20-25, wherein the method comprises:
obtaining (S116), the scanning data from an ultrasound scanning machine via an image acquiring device.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that the Figures comprise some circuitries or operations which are illustrated with a solid line and some circuitries, components, features, or operations which are illustrated with a dashed line. Circuitries or operations which are comprised in a solid line are circuitries, components, features or operations which are comprised in the broadest example. Circuitries, components, features, or operations which are comprised in a dashed line are examples which may be comprised in, or a part of, or are further circuitries, components, features, or operations which may be taken in addition to circuitries, components, features, or operations of the solid line examples. It should be appreciated that these operations need not be performed in order presented. Furthermore, it should be appreciated that not all of the operations need to be performed. The example operations may be performed in any order and in any combination. It should be appreciated that these operations need not be performed in order presented. Circuitries, components, features, or operations which are comprised in a dashed line may be considered optional.

Other operations that are not described herein can be incorporated in the example operations. For example, one or more additional operations can be performed before, after, simultaneously, or between any of the described operations.

Certain features discussed above as separate implementations can also be implemented in combination as a single implementation. Conversely, features described as a single implementation can also be implemented in multiple implementations separately or in any suitable sub-combination. Moreover, although features may be described above as acting in certain combinations, one or more features from a claimed combination can, in some cases, be excised from the combination, and the combination may be claimed as any sub-combination or variation of any sub-combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the examples may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents.

### LIST OF REFERENCES

1 user
2 imaging system
4 input
6 tissue sample representation, output
10 imaging device
10A one or more interfaces
10B memory
10C processing unit
10D image acquiring device
12 control, transmit
14 obtain
20 scanning machine
20A scanning probe
20B one or more interfaces
20C gripping element
20D, 20D1, 20D2, 20D3, 20D4 slider element
22 carrier element
23 carrier element mover
24 beam/robotic arm
26 sample area
200 tissue sample
210 first part
220 second part
230 resection margin
240 first image frame
242 second image frame
244 third image frame
250 tissue sample representation
260 reference coordinate system
270 last image frame
300 reference element
100 method for characterizing a tissue sample and a reference element
S102 obtaining scanning data
S102A obtaining scanning data from an ultrasound scanning machine via an image acquiring device
S104 obtaining image data
S106 obtaining positioning data of the reference element
S108 determining, based on the positioning data, a reference coordinate system
S110 determining a position associated with the image frame for provision of a set of positions for the plurality of image frames
S112 determining a tissue sample representation
S112A determining a first part of the tissue sample, and a second part of the tissue sample
S112B determining a first volume of the first part and a second volume of the second part
S114 determining a resection margin of the tissue sample

1-27. (canceled)

28. An imaging system for three-dimensional ultrasound imaging comprising:
an ultrasound scanning machine comprising an ultrasound scanning probe for scanning a tissue sample;
a carrier element comprising a reference element having a three-dimensional grid structure, one or more dimensions of the three-dimensional grid structure being known to the imaging device;
a cabinet comprising a first, a second, and a third slider element, for moving the ultrasound scanning probe relative to the cabinet
a processing unit;
an interface; and
wherein the processing unit is configured to:
obtain, from the ultrasound scanning machine, via the interface, scanning data indicative of a tissue sample placed in the carrier element and the reference element;
obtain, based on the scanning data, image data, where the image data is indicative of the tissue sample and the reference element; and
determine, based on the image data, a tissue sample representation and positioning data of the reference element, and to determine, based on the positioning data, a reference coordinate system.

29. The imaging system according to claim 28, wherein the image data comprises a plurality of image frames, and wherein the processing unit is configured to determine, for each image frame of the plurality of image frames, based on the reference coordinate system, a position associated with the image frame for provision of a set of positions for the plurality of image frames, and wherein the determination of the tissue sample representation is based on the set of positions.

30. The imaging system according to claim 28, wherein the determination of the tissue sample representation comprises to determine, based on the image data, a first part of the tissue sample, the first part being associated with a first type of tissue, and a second part of the tissue sample, the second part being associated with a second type of tissue.

31. The imaging system according to claim 30, wherein the processing unit is configured to determine, based on the set of positions, a first volume of the first part and a second volume of the second part.

32. The imaging system according to claim 28, wherein the processing unit is configured to determine, based on the tissue sample representation, a resection margin of the tissue sample.

33. The imaging system according to claim 28, wherein the imaging device comprises an image data acquiring device, and wherein the processing unit is configured to obtain the scanning data from the ultrasound scanning machine via the image acquiring device.

34. The imaging system according to claim 28, further comprising a fourth slider element for moving the carrier element relative to the cabinet.

35. The imaging system according to claim 28, wherein the system comprises a gripping element for attaching the ultrasound scanning probe to a slider element for moving the ultrasound scanning probe.

36. The imaging system according to claim 28, wherein the processing unit is configured to control the first, second, third, and/or fourth slider element.

37. A method, performed using an imaging system for three-dimensional ultrasound imaging according to claim 28, for characterizing a tissue sample and a reference element, the method comprising:
obtaining, from the ultrasound scanning machine of the imaging system, scanning data indicative of the tissue sample and the reference element,
obtaining, based on the scanning data, image data, where the image data is indicative of the tissue sample and the reference element; and
determining a tissue sample representation and positioning data of the reference element based on the image data, and to determine, based on the positioning data, a reference coordinate system.

38. The method according to claim 37, wherein the method comprises:
obtaining, based on the image data, positioning data of the reference element, and
determining, based on the positioning data, a reference coordinate system.

39. The method according to claim 38, wherein the image data comprises a plurality of image frames, the method comprising:
determining, for each image frame of the plurality of image frames, based on the reference coordinate system, a position associated with the image frame for provision of a set of positions for the plurality of image frames; and
where the determination of the tissue sample representation is based on the set of positions.

40. The method according to claim 37, wherein the method comprises:
determining, based on image data, a first part of the tissue sample, the first part being associated with a first type of tissue, and a second part of the tissue sample, the second part being associated with a second type of tissue.

41. The method according to claim 40, wherein the method comprises:
determining, based on the set of positions, a first volume of the first part and a second volume of the second part.

42. The method according to claim 37, wherein the method comprises:
determining, based on the tissue sample representation, a resection margin of the tissue sample.

43. The method according to claim 37, wherein the method comprises:
obtaining the scanning data from an ultrasound scanning machine via an image acquiring device.
